(19) United States
(12) Patent Application Publication
(10) Pub. No.: US 2009/0028936 A1
(43) Pub. Date: Jan. 29, 2009

(54) Tablets containing enrofloxacin and flavouring agents and/or flavours

(75) Inventors: Venkata-Rangarao Kanikanti, Leverkusen (DE); Sabine Bongaerts, Leverkusen (DE)

Correspondence Address: BAYER HEALTHCARE LLC, P.O.BOX 390, SHAWNEE MISSION, KS, 66201, US

(73) Assignee: Bayer HealthCare AG, Leverkusen (DE)

(21) Appl. No.: 10/559,995

(22) PCT Filed: Jun. 14, 2004
(86) PCT No.: PCT/EP04/06370

§ 371 Date: Aug. 7, 2007

(51) Int. Cl. A61K 31/47; A61K 9/20

(52) U.S. Cl. 424/464; 514/311

(57) ABSTRACT

The present invention relates to tablets for animals, which tablets comprise enrofloxacin as well as flavourings and/or aromatizing substances.

The present invention relates to tablets for animals, which tablets comprise enrofloxacin as well as flavourings and/or aromatizing substances.

Administering tablets to animals constitutes a problem since the tablets are in no way attractive to the animals and are as a rule only ingested involuntarily by them. Usually, the tablets have to be packaged in feed in order to administer them. When this is done, it is not always guaranteed that the medicine can be administered completely and consequently in the correct dosage. The release profile of the pharmaceutical can also be changed when it is administered in the feed.

It is in principle already known that palatability can be increased by adding suitable aromas and/or flavourings. However, adding these substances frequently impairs the mechanical properties of the tablets to a degree which is unacceptable in practice.

There is therefore a need for readily palatable tablets which possess acceptable mechanical properties.

The invention relates to:

Tables comprising:

from 20 to 45% by weight of enrofloxacin
from 18 to 35% by weight of lactose
from 5 to 10% by weight of microcrystalline cellulose, and
from 5 to 20% by weight of meat flavour.

The values in percent by weight are based on the total weight of the tablet.

Enrofloxacin is used in a quantity of from 20 to 45% by weight, preferably of from 23 to 42% by weight.

Enrofloxacin carries the systematic designation 1-cyclopropyl-7-(4-ethyl-1-piperazinyl)-6-fluoro-1,4-dihydro-4-oxo-3-quinolinecarboxylic acid and has the following structural formula:

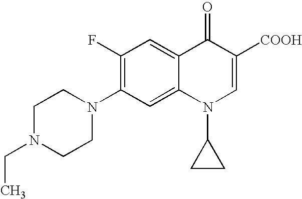

According to the invention, enrofloxacin can also be used in the form of its pharmaceutically utilizable salts and hydrates.

Suitable salts are pharmaceutically utilizable acid addition salts and basic salts.

Pharmaceutically utilizable salts are to be understood as being, for example, the salts of hydrochloric acid, sulphuric acid, acetic acid, glycolic acid, lactic acid, succinic acid, citric acid, tartaric acid, methanesulphonic acid, 4-toluenesulphonic acid, galacturonic acid, gluconic acid, embonic acid, glutamic acid and aspartic acid. In addition, enrofloxacin can also be bonded to acidic or basic ion exchangers. Pharmaceutically utilizable basic salts which may be mentioned are the alkali metal salts, for example the sodium salts or potassium salts, the alkaline earth metal salts, for example the magnesium salts or calcium salts, the zinc salts, the silver salts and the guanidinium salts.

Hydrates are understood as meaning both the hydrates of enrofloxacin itself and the hydrates of its salts.

Lactose is a commercially available pharmaceutical adjuvant which can be obtained in a variety of forms, e.g. spray-dried or as anhydrous lactose. According to the invention, preference is given to using lactose monohydrate (e.g. Milchzucker fein [fine-quality lactose] from DMV International). The tablets according to the invention comprise from 18 to 35% by weight of lactose, preferably from 19 to 30% by weight, based on the total weight of the tablet.

Microcrystalline cellulose is a commercially available pharmaceutical adjuvant (e.g. Avicel® PH 101 from FMC). The tablets according to the invention comprise from 5 to 10% by weight, preferably from 5.5 to 8% by weight, based on the total weight of the tablet.

Dry liver powders from cattle, poultry, sheep or pigs, preferably from poultry and pigs, as well as other flavour preparations, are suitable for use as a meat flavour. The flavours which are commercially available under the designations Artificial Beef Flavor and BAYOPAL®, and which are supplied by the companies Pharma Chemie (Artificial Beef Flavor) and Haarmann and Reimer (BAYOPAL®) are very particularly suitable.

The meat flavour is preferably used in a quantity of from 5% to 20%, preferably of from 7% to 15%, particularly preferably of from 9% to 11%. In this connection, the figures in percent are percentages by weight of the finished tablet.

In addition to the abovementioned ingredients, the tablets according to the invention can also comprise further customary pharmaceutical excipients and adjuvants.

All physiologically tolerated solid inert substances may be mentioned as excipients. Inorganic and organic substances may be used for this purpose. Examples of inorganic substances are sodium chloride, carbonates, such as calcium carbonate, hydrogen carbonates, aluminium oxides, silicic acids, argillaceous earths, precipitated or colloidal silicon dioxide and phosphates.

The tablets according to the invention preferably comprise silicon dioxide, in particular colloidal anhydrous silicon dioxide, in quantities of from 0.05 to 0.3% by weight, in particular of from 0.1 to 0.2% by weight, based on the total weight of the tablet.

Examples of organic substances are sugars, cellulose, foodstuffs and feedstuffs such as milk powder, carcass meals, flours and coarse meals, and starches.

The tablets according to the invention preferably comprise starch, such as maize starch, as an additional excipient, specifically in quantities of usually from 10 to 40% by weight, preferably of from 15 to 30% by weight, particularly preferably of from 18 to 26% by weight, based on the total weight of the tablet.

The tablets can comprise additional customary pharmaceutical adjuvants. Those which may be mentioned by way of example are: lubricants and glidants, such as magnesium stearate, stearic acid, talc and bentonites; disintegration-promoting substances such as starch, crosslinked sodium carboxymethyl cellulose or crosslinked polyvinylpyrrolidone; binders, such as starch, gelatine, cellulose ether or linear polyvinylpyrrolidone, and also dry binders such as microcrystalline cellulose.

The tablets according to the invention preferably comprise a lubricant, in particular magnesium stearate, in quantities of from 0.4 to 1.0% by weight, preferably of from 0.5 to 0.8% by weight, based on the total weight of the tablet.

The tablets according to the invention preferably comprise a binder, in particular a polyvinylpyrrolidone (e.g. polyvidone), in quantities of from 1.5 to 4% by weight, preferably of from 2 to 3% by weight, based on the total weight of the tablet.

The tablets according to the invention can be produced by means of a process in which
(a) Enrofloxacin, lactose, where appropriate meat flavour and also, where appropriate, additional adjuvants are mixed,
(b) the mixture is granulated in the added presence of water or aqueous solutions of additional adjuvants,
(c) this mixture is dried,
(d) after drying, microcrystalline cellulose and, where appropriate, additional adjuvants and also meat flavour, provided this was not added in step (a), are admixed,
(e) and the mixture is subsequently pressed into tablets.

Starch, in particular maize starch, is preferably added as an additional adjuvant in step (a). It is particularly advantageous only to add a portion of the total quantity of starch employed at this point.

An aqueous solution of polyvinylpyrrolidone is preferably added as an additional adjuvant in step (b).

In connection with the drying in step (c), it is found to be advantageous to keep to a residual moisture of less than 5%, preferably of from 1 to 4% (determined as loss on drying).

Starch, colloidal silicon dioxide and magnesium stearate are preferably added as additional adjuvants in step (d). In so far as a portion of the starch was already added in step (a), the second portion of the total quantity is admixed in step (d).

The antibiotic spectrum of action of enrofloxacin is known. The pharmaceuticals according to the invention are therefore suitable for the prophylaxis and treatment of corresponding bacterial diseases and diseases which are caused by bacterium-like organisms. The compositions according to the invention are generally suitable for use in animal husbandry and animal breeding in the case of productive animals, breeding animals, zoo animals, laboratory animals, experimental animals and pet animals. Preference is naturally given to using them in the case of those animals where adding the meat flavour can be expected to improve the palatability.

The productive animals and breeding animals include mammals, such as cattle, horses, sheep, pigs, goats, camels, water buffaloes, donkeys, rabbits, fallow deer, reindeer and fur animals such as mink, chinchilla and raccoon.

Laboratory animals and experimental animals include mice, rats, guinea pigs, golden hamsters, dogs and cats.

The pet animals include dogs and cats.

The compositions according to the invention are particularly preferably used for dogs and cats, in particular dogs.

The bacterial diseases in animals include, for example, swine dysentery; leptospirosis in cattle, pigs, horses and dogs; Campylobacter enteritis in cattle; Campylobacter abortion in sheep and pigs; infections of the skin; pyodermas in dogs; otitis externa; mastitis in cattle, sheep and goats; streptococcal mastitis; streptococcal infection in horses, in pigs and in other animal species; pneumococcal infection in calves and in other animal species; glanders; conjunctivitis; enteritides; pneumonias; brucellosis in cattle, sheep and pigs; atrophic rhinitis in pigs; salmonellosis in cattle, horses, sheep and other animal species; septicaemias; Escherichia coli infection in piglets; metritis-mastitis-agalactia (MA) Syndrome; Klebsiella infections; pseudotuberculosis; infectious pleuropneumonia; primary pasteurelloses; joint ill; necrobacillosis in cattle and in domestic animals; leptospirosis; erysipelas in pigs and other animal species, listeriosis; anthrax, clostridioses; tetanus infections, botulism; infections with Corynebacterium pyogenes; tuberculosis in cattle, sheep and other animal species; paratuberculosis in ruminants; nocardiosis; Q fever; ornithosis-psittacosis; encephalomyelitis; mycoplasmosis in cattle and other animals, enzootic pneumonia in pigs.

The tablets according to the invention have a comparatively low hardness (e.g. the tablet described in example (1) has a diameter of 5 mm and hardness in the order of size of 20-30 N); this is a known problem in tablets to which flavours have been added. Surprisingly, the tablets according to the invention are characterized by an abrasion resistance which is relatively high in comparison with their low hardness, which means that they can readily be used in practice. The pharmacopoeias (e.g. Ph Eur or USP) describe methods for testing, and minimum requirements for, the abrasion resistance of tablets.

### EXAMPLES

| | (1) | (2) | (3) |
|---|---|---|---|
| Ingredients | mg | mg | mg |
| Enrofloxacin | 15.00 | 50.00 | 150.00 |
| Lactose monohydrate | 17.80 | 23.60 | 100.40 |
| Maize starch | 15.20 | 22.40 | 86.10 |
| Microcrystalline cellulose | 4.00 | 8.00 | 28.00 |
| Polyvidone | 1.50 | 3.00 | 10.00 |
| Magnesium stearate | 0.40 | 0.80 | 2.80 |
| Anhydrous colloidal | 0.10 | 0.20 | 0.70 |
| silicon dioxide | | | |
| Irradiated artificial beef | 6.00 | 12.00 | 42.00 |
| flavour | | | |
| Tablet weight | 60.00 | 120.00 | 420.00 |

1. Tablets comprising:
from 20 to 45% by weight of enrofloxacin
from 18 to 35% by weight of lactose
from 5 to 10% by weight of microcrystalline cellulose, and
from 5 to 20% by weight of meat flavour.

2. Process for producing tablets according to claim 1, in which
(a) Enrofloxacin, lactose, where appropriate meat flavour and also, where appropriate, additional adjuvants are mixed,
(b) the mixture is granulated in the added presence of water or aqueous solutions of additional adjuvants,
(c) this mixture is dried,
(d) after drying, microcrystalline cellulose and, where appropriate, additional adjuvants and also meat flavour, provided this was not added in step (a), are admixed,
(e) and the mixture is subsequently pressed into tablets.
